(19) United States
(12) Patent Application Publication
(10) Pub. No.: US 2006/0204562 A1
(43) Pub. Date: Sep. 14, 2006

(54) Microprojection arrays with improved biocompatibility

(76) Inventors: Michel J.N. Cormier, Mountain View, CA (US); WeiQi Lin, Palo Alto, CA (US); Cynthia Zhang, Saratoga, CA (US); Ahmad Parsa Samiee, Fremont, CA (US); Rolfe Anderson, Saratoga, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/355,729

(22) Filed: Feb. 15, 2006

Related U.S. Application Data
(60) Provisional application No. 60/653,675, filed on Feb. 16, 2005.

(51) Int. Cl. A61F 13/02; A61M 31/00; A61L 15/16

(52) U.S. Cl. 424/448; 604/500

(57) ABSTRACT

A transdermal delivery member having a plurality of microprojections adapted to pierce the stratum corneum of a subject, each of the microprojection having a length less than 145 microns. In a preferred embodiment, each microprojection has a length in the range of approximately 50-145 microns.

### CROSS-REFERENCE TO RELATED APPLICATIONS

This application claims the benefit of U.S. Provisional Application No. 60/653,675, filed Feb. 16, 2005.

### FIELD OF THE PRESENT INVENTION

The present invention relates generally to transdermal agent delivery systems and methods. More particularly, the invention relates to transdermal agent delivery systems having microprojection arrays with improved biocompatibility.

### BACKGROUND OF THE INVENTION

Active agents or drugs are typically administered either orally or by injection. Unfortunately, many active agents are completely ineffective or have radically reduced efficacy when orally administered, since they either are not absorbed or are adversely affected before entering the bloodstream and, hence, do not possess the desired activity. On the other hand, the direct injection of the active agent into the bloodstream, while assuring no modification of the agent during administration, is a difficult, inconvenient, painful and uncomfortable procedure, which often results in poor patient compliance.

Hence, in principle, transdermal delivery provides for a method of administering active agents that would otherwise need to be delivered orally or via hypodermic injection or intravenous infusion. Transdermal drug delivery offers improvements in both of these areas. Transdermal delivery, when compared to oral delivery, avoids the harsh environment of the digestive tract, bypasses gastrointestinal drug metabolism, reduces first-pass effects, and avoids the possible deactivation by digestive and liver enzymes. Transdermal delivery is also a relatively simple, convenient and virtually painless procedure.

The word “transdermal” is used herein as a generic term referring to passage of an agent across the skin layers. The word “transdermal” refers to delivery of an agent (e.g., a therapeutic agent, such as a drug or an immunologically active agent, such as a vaccine) into and/or through the skin to the local tissue or systemic circulatory system without substantial cutting or penetration of the skin, such as cutting with a surgical knife or piercing the skin with a hypodermic needle. Transdermal agent delivery includes delivery via passive diffusion as well as delivery based on external energy sources, including electricity (e.g., iontophoresis) and ultrasound (e.g., phonophoresis).

As is well known in the art, transdermal agent flux is dependent upon the condition of the skin, the size and physical/chemical properties of the agent molecule, and the concentration gradient across the skin. This low permeability is attributed primarily to the stratum corneum, the outermost skin layer, which consists of flat, dead cells filled with keratin fibers (keratinocytes) surrounded by lipid bilayers. This highly-ordered structure of the lipid bilayers confers a relatively impermeable character to the stratum corneum.

Various pretreatment methods and apparatus have thus been employed to enhance the transdermal drug flux. Illustrative are the methods and apparatus disclosed in U.S. Pat. Nos. 3,918,449, 5,611,806 and 5,964,729.

There are, however, numerous drawbacks and disadvantages associated with the disclosed prior art pretreatment methods and apparatus. Among the drawbacks are that most of the devices employ one or more “rolling structures” that are adapted to pierce the skin via manual force. As a result, there are significant variations in the effected (or pretreated) area from patient to patient. Variations in the force applied and, hence, penetration of the piercing elements are also likely by virtue of the differences in strength and/or applied angle of the device from patient to patient.

Other systems and apparatus that employ tiny skin piercing elements or microprojections to enhance transdermal agent delivery are disclosed in European Patent EP 0 407063A1, U.S. Pat. Nos. 5,879,326, 3,814,097, 5,279,54, 5,250,023, 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated by reference in their entirety.

The disclosed systems include an integral reservoir for holding the active agent and also a delivery system to transfer the agent from the reservoir through the stratum corneum, such as by hollow tines of the device itself. One example of such a device is disclosed in WO 93/17754, which has a liquid drug reservoir. The reservoir must, however, be pressurized to force the liquid drug through the tiny tubular elements and into the skin. Disadvantages of such devices thus include the added complication and expense for adding a pressurizable liquid reservoir and complications due to the presence of a pressure-driven delivery system.

In U.S. application Ser. Nos. 10/637,909, 10/880,702, 10/911,299, 10/971,430, 10/970,901, 10/971,224, 10/972,231, 10/971,338, 10/559,153 and 60/585,276, which are incorporated by reference herein in their entirety, further systems and apparatus that employ microprojections to enhance transdermal agent flux are disclosed. Some of the noted systems and apparatus include an agent-containing biocompatible coating that is disposed on the microprojections. Upon application of the microprojections to the skin of a subject, the microprojections pierce the stratum corneum and the agent-containing coating is dissolved by body fluid (i.e., intracellular fluids and extracellular fluids, such as interstitial fluid). The dissolved coating is then released into the skin (i.e., bolus delivery) for systemic delivery. In other noted systems, instead if being coated on the microprojections, the biologically active agent is included in a gel pack or a dry film.

The disclosed systems and apparatus employ microprojections of various shapes and sizes to pierce the stratum corneum of the skin. The microprojections generally extend perpendicularly from a thin, flat member, such as a pad or sheet.

The microprojections disclosed in the noted references generally have a length less than 500 microns, in some instances, less than 250 microns. However, the references do not teach or suggest a range of microprojection length that provides optimal biocompatibility.

It is therefore an object of the present invention to provide a transdermal agent delivery apparatus and system that substantially reduces or eliminates the aforementioned drawbacks and disadvantages associated with prior art agent delivery systems.

It is another object of the present invention to provide a transdermal agent delivery apparatus and system that enhances transdermal agent delivery.

It is another object of the present invention to provide a transdermal agent delivery member having optimal biocompatibility.

It is another object of the present invention to provide a microprojection array adapted to pierce the stratum corneum having optimal biocompatibility.

It is another object of the present invention to provide a microprojection array that, when applied to the skin of a subject, does not produce appreciable bleeding.

It is yet another object of the present invention to provide a microprojection array that, when applied to the skin of a subject, does not produce appreciable irritation.

### SUMMARY OF THE INVENTION

In accordance with the above objects and those that will be mentioned and will become apparent below, the transdermal delivery member of the invention includes a plurality of microprojections adapted to pierce the stratum corneum of a subject, each of the microprojection having a length in the range of approximately 50-145 microns.

Preferably, each microprojection has a length in the range of approximately 70-140 microns.

Preferably, the microprojections are arranged in an array, the array having a microprojection density greater than 100 microprojections/cm².

In one embodiment, the microprojections are constructed out of stainless steel, titanium, nickel titanium alloys, or similar biocompatible materials, such as polymeric materials.

In another embodiment, the microprojections are constructed out of a non-conductive material, such as a polymer.

In one embodiment of the invention, the delivery member includes a biocompatible coating having at least one biologically active agent. Preferably, the agent-containing biocompatible coating is disposed on the microprojections.

In one embodiment, the biologically active agent is selected from the group consisting of small molecular weight compounds, polypeptides, proteins, oligonucleotides, nucleic acids and polysaccharides.

In another embodiment, the biologically active agent is selected from the group consisting of ACTH, amylin, angiotensin, angiogenin, anti-inflammatory peptides, BNP, calcitonin, endorphins, endothelin, GLIP, Growth Hormone Releasing Factor (GRF), hirudin, insulin, insulinotropin, neuropeptide Y, PTH, VIP, growth hormone release hormone (GHRH), octreotide, pituitary hormones (e.g., hGH), ANF, growth factors, such as growth factor releasing factor (GFRF), bMSH, somatostatin, platelet-derived growth factor releasing factor, human chorionic gonadotropin, erythropoietin, glucagon, hirulog, interferon alpha, interferon beta, interferon gamma, interleukins, granulocyte macrophage colony stimulating factor (GM-CSF), granulocyte colony stimulating factor (G-CSF), menotropins (urofollitropin (FSH) and LH)), streptokinase, tissue plasminogen activator, urokinase, ANF, ANP, ANP clearance inhibitors, antidiuretic hormone agonists, calcitonin gene related peptide (CGRP), IGF-1, pentigetide, protein C, protein S, thymosin alpha-1, vasopressin antagonists analogs, alpha-MSH, VEGF, PYY, fondaparinux, ardeparin, dalteparin, defibrotide, enoxaparin, hirudin, nadroparin, reviparin, tinzaparin, pentosan polysulfate, oligonucleotides and oligonucleotide derivatives, such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and analogs and derivatives derived from the foregoing and mixtures thereof.

In an alternative embodiment, the biologically active agent comprises a formulation having an immunologically active agent selected from the group consisting of proteins, polysaccharide conjugates, oligosaccharides, lipoproteins, subunit vaccines, Bordetella pertussis (recombinant PT accince—acellular), Clostridium tetani (purified, recombinant), Corynebacterium diphtheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), Vibrio cholerae (conjugate lipopolysaccharide), whole virus, bacteria, weakened or killed viruses, cytomegalo virus, hepatitis B virus, hepatitis C virus, human papillomavirus, rubella virus, varicella zoster, weakened or killed bacteria, bordetella pertussis, clostridium tetani, corynebacterium diphtheriae, group A streptococcus, legionella pneumophila, neisseria meningitidis, pseudomonas aeruginosa, streptococcus pneumoniae, treponema pallidum, vibrio cholerae, flu vaccines, lyme disease vaccine, rabies vaccine, measles vaccine, mumps vaccine, chicken pox vaccine, small pox vaccine, hepatitis vaccine, pertussis vaccine, diphtheria vaccine, nucleic acids, single-stranded and double-stranded nucleic acids, supercoiled plasmid DNA, linear plasmid DNA, cosmids, bacterial artificial chromosomes (BACs), yeast artificial chromosomes (YACs), mammalian artificial chromosomes, and RNA molecules.

The method for delivering a biologically active agent through the skin of a patient, in accordance with one embodiment of the invention, comprises the steps of (i) providing a transdermal delivery member having a plurality of microprojections that define a microprojection array, each of the microprojections having a length in the range of approximately 50-145 microns, the delivery member including an agent-containing biocompatible coating, and (ii) applying the microprojection to the skin of a subject.

In a preferred embodiment, each microprojection has a length in the range of approximately 70-140 microns.

Preferably, the microprojection array has a microprojection density greater than 100 microprojections/cm².

In one embodiment of the invention, the agent-containing biocompatible coating includes at least one biologically active agent, the biologically active agent being selected from the group consisting of small molecular weight compounds, polypeptides, proteins, oligonucleotides, nucleic acids and polysaccharides.

In another embodiment, the biologically active agent is selected from the group consisting of ACTH, amylin, angiotensin, angiogenin, anti-inflammatory peptides, BNP, calcitonin, endorphins, endothelin, GLIP, Growth Hormone Releasing Factor (GRF), hirudin, insulin, insulinotropin, neuropeptide Y, PTH, VIP, growth hormone release hormone (GHRH), octreotide, pituitary hormones (e.g., hGH), ANF, growth factors, such as growth factor releasing factor (GFRF), bMSH, somatostatin, platelet-derived growth factor releasing factor, human chorionic gonadotropin, erythropoietin, glucagon, hirulog, interferon alpha, interferon beta, interferon gamma, interleukins, granulocyte macrophage colony stimulating factor (GM-CSF), granulocyte colony stimulating factor (G-CSF), menotropins (urofollitropin (FSH) and LH)), streptokinase, tissue plasminogen activator, urokinase, ANF, ANP, ANP clearance inhibitors, antidiuretic hormone agonists, calcitonin gene related peptide (CGRP), IGF-1, pentigetide, protein C, protein S, thymosin alpha-1, vasopressin antagonists analogs, alpha-MSH, VEGF, PYY, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen , alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and analogs and derivatives derived from the foregoing and mixtures thereof.

In an alternative embodiment, the biologically active agent comprises a formulation having an immunologically active agent selected from the group consisting of proteins, polysaccharide conjugates, oligosaccharides, lipoproteins, subunit vaccines, Bordetella pertussis (recombinant PT accince—acellular), Clostridium tetani (purified, recombinant), Corynebacterium diphtheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), Vibrio cholerae (conjugate lipopolysaccharide), whole virus, bacteria, weakened or killed viruses, cytomegalo virus, hepatitis B virus, hepatitis C virus, human papillomavirus, rubella virus, varicella zoster, weakened or killed bacteria, bordetella pertussis, clostridium tetani, corynebacterium diphtheriae, group A streptococcus, legionella pneumophila, neisseria meningitidis, pseudomonas aeruginosa, streptococcus pneumoniae, treponema pallidum, vibrio cholerae, flu vaccines, lyme disease vaccine, rabies vaccine, measles vaccine, mumps vaccine, chicken pox vaccine, small pox vaccine, hepatitis vaccine, pertussis vaccine, diphtheria vaccine, nucleic acids, single-stranded and double-stranded nucleic acids, supercoiled plasmid DNA, linear plasmid DNA, cosmids, bacterial artificial chromosomes (BACs), yeast artificial chromosomes (YACs), mammalian artificial chromosomes, and RNA molecules.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a partial perspective view of one embodiment of a microprojection array, according to the invention;

FIG. 2 is a partial perspective view of one embodiment of a microprojection array having a biocompatible coating disposed on the microprojections, according to the invention;

FIG. 3 illustrates microprojection designs with increasing length that can be employed within the scope of the invention;

FIG. 4 is a series of photographs of a skin site after application of microprojections having a length ≧145 microns;

FIG. 5 is a series of photographs of a skin site after application of microprojections having a length ≧145 microns;

FIG. 6 is a graph showing the combined erythema+edema score obtained with various microprojection designs;

FIG. 7 is a graph showing agent absorption as a function of microprojection length; and

FIG. 8 is a graph of agent delivery as a function of time for a microprojection design of the invention.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a”, “an” and “the” include plural referents unless the content clearly dictates otherwise. Thus, for example, reference to “an active agent” includes two or more such agents; reference to “a microprojection” includes two or more such microprojections and the like.

### Definitions

The term “transdermal”, as used herein, means the delivery of an agent into and/or through the skin for local or systemic therapy.

The term “transdermal flux”, as used herein, means the rate of transdermal agent delivery.

The term “co-delivering”, as used herein, means that a supplemental agent(s) is administered transdermally either before the agent is delivered, before and during transdermal flux of the agent, during transdermal flux of the agent, during and after transdermal flux of the agent, and/or after transdermal flux of the agent.

The term “biologically active agent”, as used herein, refers to a composition of matter or mixture containing an active agent that is pharmacologically effective when administered in a therapeutically effective amount. Examples of such active agents include, without limitation, small molecular weight compounds, polypeptides, proteins, oligonucleotides, nucleic acids and polysaccharides.

Further examples of “biologically active agents” include, without limitation, ACTH, amylin, angiotensin, angiogenin, anti-inflammatory peptides, BNP, calcitonin, endorphins, endothelin, GLIP, Growth Hormone Releasing Factor (GRF), hirudin, insulin, insulinotropin, neuropeptide Y, PTH, VIP, growth hormone release hormone (GHRH), , octreotide, pituitary hormones (e.g., hGH), ANF, growth factors, such as growth factor releasing factor (GFRF), bMSH, somatostatin, platelet-derived growth factor releasing factor, human chorionic gonadotropin, erythropoietin, glucagon, hirulog, interferon alpha, interferon beta, interferon gamma, interleukins, granulocyte macrophage colony stimulating factor (GM-CSF), granulocyte colony stimulating factor (G-CSF), menotropins (urofollitropin (FSH) and LH)), streptokinase, tissue plasminogen activator, urokinase, ANF, ANP, ANP clearance inhibitors, antidiuretic hormone agonists, calcitonin gene related peptide (CGRP), IGF-1, pentigetide, protein C, protein S, thymosin alpha-1, vasopressin antagonists analogs, alpha-MSH, VEGF, PYY, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and analogs and derivatives derived from the foregoing and mixtures thereof.

The noted biologically active agents can also be in various forms, such as free bases, acids, charged or uncharged molecules, components of molecular complexes or nonirritating, pharmacologically acceptable salts. Further, simple derivatives of the active agents (such as ethers, esters, amides, etc.), which are easily hydrolyzed at body pH, enzymes, etc., can be employed.

The term “biologically active agent”, as used herein, also refers to a composition of matter or mixture containing a “vaccine” or other immunologically active agent or an agent that is capable of triggering the production of an immunologically active agent, and which is directly or indirectly immunologically effective when administered in an immunologically effective amount.

The term “vaccine”, as used herein, refers to conventional and/or commercially available vaccines, including, but not limited to, Bordetella pertussis (recombinant PT accince—acellular), Clostridium tetani (purified, recombinant), Corynebacterium diphtheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), Vibrio cholerae (conjugate lipopolysaccharide), whole virus, bacteria, weakened or killed viruses, cytomegalo virus, hepatitis B virus, hepatitis C virus, human papillomavirus, rubella virus, varicella zoster, weakened or killed bacteria, bordetella pertussis, clostridium tetani, corynebacterium diphtheriae, group A streptococcus, legionella pneumophila, neisseria meningitidis, pseudomonas aeruginosa, streptococcus pneumoniae, treponema pallidum, vibrio cholerae, flu vaccines, lyme disease vaccine, rabies vaccine, measles vaccine, mumps vaccine, chicken pox vaccine, small pox vaccine, hepatitis vaccine, pertussis vaccine, diphtheria vaccine, nucleic acids, single-stranded and double-stranded nucleic acids, supercoiled plasmid DNA, linear plasmid DNA, cosmids, bacterial artificial chromosomes (BACs), yeast artificial chromosomes (YACs), mammalian artificial chromosomes, and RNA molecules.

It is to be understood that more than one biologically active agent can be employed within the scope of this invention, and that the use of the term “biologically active agent” (or “active agent”) in no way excludes the use of two or more such active agents.

The term “biologically effective amount” or “biologically effective rate” shall be used when the biologically active agent is a pharmaceutically active agent and refers to the amount or rate of the pharmacologically active agent needed to effect the desired therapeutic, often beneficial, result. The amount of active agent employed will be that amount necessary to deliver a therapeutically effective amount of the active agent to achieve the desired therapeutic result. In practice, this will vary widely depending upon the particular pharmacologically active agent being delivered, the site of delivery, the severity of the condition being treated, the desired therapeutic effect and the release kinetics for delivery of the agent from the hydrogel into skin tissues.

The term “biologically effective amount” or “biologically effective rate” shall also be used when the biologically active agent is an immunologically active agent and refers to the amount or rate of the immunologically active agent needed to stimulate or initiate the desired immunologic, often beneficial result. The amount of the immunologically active agent employed will be that amount necessary to deliver an amount of the active agent needed to achieve the desired immunological result. In practice, this will similarly vary widely depending upon the particular immunologically active agent being delivered, the site of delivery, and the dissolution and release kinetics for delivery of the active agent into skin tissues.

The term “microprojections”, as used herein, refers to piercing elements that are adapted to pierce or cut through the stratum corneum into the underlying epidermis layer, or epidermis and dermis layers, of the skin of a living animal, particularly a mammal and more particularly a human.

As discussed in detail herein, in one embodiment of the invention, the microprojections preferably have a projection length less than 145 microns, more preferably, in the range of approximately 50-145 microns, even more preferably, in the range of approximately 70-140 microns.

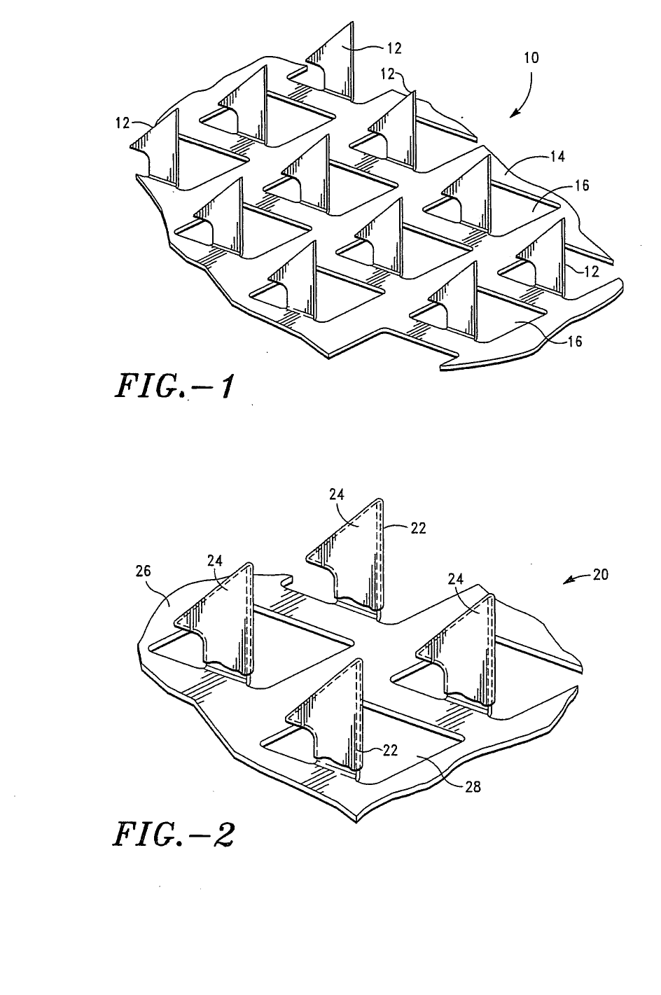

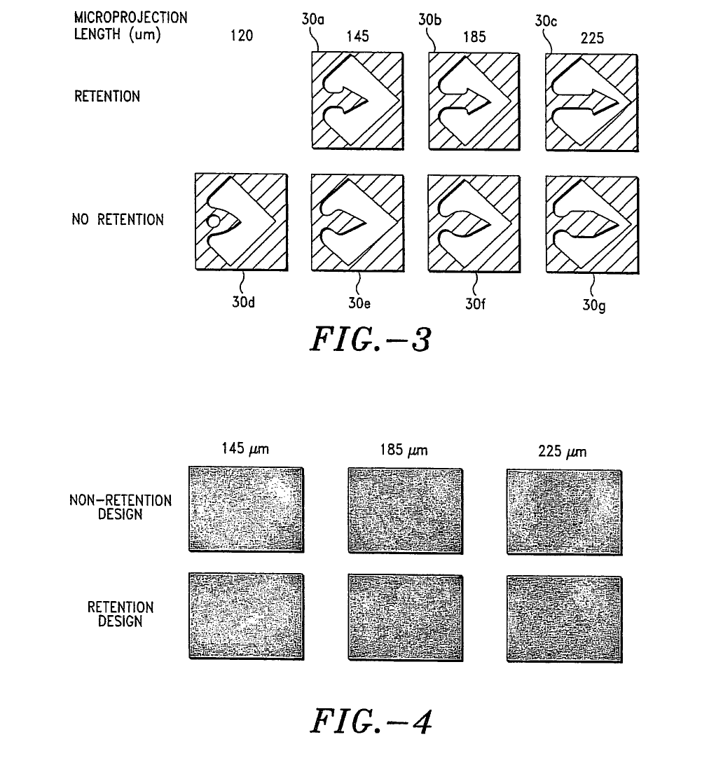

The terms “projection length” and “length”, as used herein to describe the microprojections, mean the active length of a microprojection that pierces into the skin. Thus, in some embodiments, such as the embodiments shown in FIGS. 1 and 2, the microprojection “length” means the active length of the microprojection from the base sheet 14 to the leading tip of the microprojection. In other embodiments, wherein a microprojection stop is employed to limit the penetration depth of the microprojection, the microprojection “length” means the active length of the microprojection from the stop to the leading tip of the microprojection.

The microprojections of the invention preferably have an average width (average of width and thickness taken at half the length of the microprojection) of about 10 μm to 100 μm. More preferably the microprojections have an average width (average of width and thickness taken at half the length of the microprojection) of about 20 μm to 80 μm.

The microprojections can be formed in different shapes, such as needles, blades, lances, pins, punches, and combinations thereof.

The term “microprojection array”, as used herein, refers to a plurality of microprojections arranged in (or defining) an array for piercing the stratum corneum. The microprojection array can be formed by etching or punching a plurality of microprojections from a thin sheet and folding or bending the microprojections out of the plane of the sheet to form a configuration, such as that shown in FIG. 1.

The microprojection array can also be formed in other known manners, such as by forming one or more strips having microprojections along an edge of each of the strip(s), as disclosed in U.S. Pat. No. 6,050,988, which is incorporated by reference herein in its entirety.

As indicated above, the present invention comprises a transdermal delivery apparatus and system that includes a plurality of microprojections adapted to pierce the stratum corneum of a subject, each of the microprojection having a length less than 145 microns, more preferably, the length is in the range of approximately 50-145 microns, more preferably, the length is in the range of approximately 70-140 microns.

Preferably, the microprojections are arranged in an array. In a preferred embodiment of the invention, the array has a microprojection density greater than 100 microprojections/cm². More preferably, the array has a microprojection density in the range of approximately 200-3000 microprojections/cm^{2.}

As discussed in detail herein, Applicants have found that the noted transdermal delivery apparatus provides optimal biocompatible. Most significantly, the microprojection arrays of the invention do not produce appreciable bleeding or irritation when applied to the skin of a subject.

As will be appreciated by one having ordinary skill in the art, the present invention has utility in connection with the delivery of biologically active agents within any of the broad class of agents normally delivered though body surfaces and membranes, including skin. In general, this includes active agents in all of the major therapeutic areas.

Referring now to FIG. 1, there is shown one embodiment of the microprojection array 10. As illustrated in FIG. 1, the microprojection array 10 includes a plurality of microprojections 12 that extend downward from one surface of a sheet or plate 14.

The microprojections 12 are generally formed from a single piece of sheet material and are sized and shaped to puncture the stratum corneum of the skin, whereby microslits are formed in the stratum corneum to enhance the transdermal agent flux. In the illustrated embodiment, the sheet 14 is formed with openings 16 disposed proximate the microprojections 12. However, according to the invention, the microprojection array 10 need not include openings 16 or any retention features. Thus, in one embodiment of the invention, the microprojection array 10 does not include openings or retainer projections.

Preferably, each microprojection 12 has a projection length less than approximately 145 microns. More preferably, each microprojection has a length in the range of approximately 50-145 microns. In a preferred embodiment, the microprojections 12 have a length in the range of approximately 70-140 microns.

According to the invention, the number of microprojections 12 in the microprojection array 10 is variable with respect to the desired flux rate, agent being sampled or delivered, delivery or sampling device used (i.e., electrotransport, passive, osmotic, pressure-driven, etc.), and other factors as will be evident to one of ordinary skill in the art. In general, the larger the number of microprojections per unit area (i.e., microprojection density), the more distributed is the flux of the agent through the skin, since there are more pathways.

Preferably, the microprojection density is at least approximately 100 microprojections/cm². In one embodiment of the invention, the microprojection density is in the range of approximately 200-3000 microprojections/cm².

In one embodiment, the microprojections 12 are constructed out of stainless steel, titanium, nickel titanium alloys, or similar biocompatible materials, such as polymeric materials.

In another embodiment, the microprojections 12 are constructed out of a non-conductive material, such as a polymer.

Further details of microprojection array 10 described above and other microprojection devices, arrays and systems that can be employed within the scope of the invention are disclosed in U.S. Pat. Nos. 6,322,808, 6,230,051 B1 and the aforementioned Co-Pending U.S. Applications, particularly, U.S. application Ser. Nos. 10/971,430 and 10/970,901, which are incorporated by reference herein in their entirety.

Referring now to FIG. 2, there is shown one embodiment of the invention, wherein the microprojections 22 of the array 20 include an agent-containing biocompatible coating 24. As illustrated in FIG. 2, the microprojections 22 similarly extend downward from a sheet 26, which has openings 28 formed therein.

According to the invention, the agent-containing coating contains at least one biologically active agent. In one embodiment of the invention, the biologically active agent is selected from the group consisting of small molecular weight compounds, polypeptides, proteins, oligonucleotides, nucleic acids and polysaccharides.

In another embodiment, the biologically active agent is selected from the group consisting of ACTH, amylin, angiotensin, angiogenin, anti-inflammatory peptides, BNP, calcitonin, endorphins, endothelin, GLIP, Growth Hormone Releasing Factor (GRF), hirudin, insulin, insulinotropin, neuropeptide Y, PTH, VIP, growth hormone release hormone (GHRH), octreotide, pituitary hormones (e.g., hGH), ANF, growth factors, such as growth factor releasing factor (GFRF), bMSH, somatostatin, platelet-derived growth factor releasing factor, human chorionic gonadotropin, erythropoietin, glucagon, hirulog, interferon alpha, interferon beta, interferon gamma, interleukins, granulocyte macrophage colony stimulating factor (GM-CSF), granulocyte colony stimulating factor (G-CSF), menotropins (urofollitropin (FSH) and LH)), streptokinase, tissue plasminogen activator, urokinase, ANF, ANP, ANP clearance inhibitors, antidiuretic hormone agonists, calcitonin gene related peptide (CGRP), IGF-1, pentigetide, protein C, protein S, thymosin alpha-1, vasopressin antagonists analogs, alpha-MSH, VEGF, PYY, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and analogs and derivatives derived from the foregoing and mixtures thereof.

In an alternative embodiment, the biologically active agent comprises one of the aforementioned vaccines.

It will be appreciated by one having ordinary skill in the art that in order to facilitate agent transport across the skin barrier, the present invention can also be employed in conjunction with a wide variety of iontophoresis or electrotransport systems, as the invention is not limited in any way in this regard. Illustrative electrotransport agent delivery systems are disclosed in U.S. Pat. Nos. 5,147,296, 5,080,646, 5,169,382 and 5,169383, the disclosures of which are incorporated by reference herein in their entirety.

The term “electrotransport” refers, in general, to the passage of a beneficial agent, e.g., a drug or drug precursor, through a body surface such as skin, mucous membranes, nails, and the like. The transport of the agent is induced or enhanced by the application of an electrical potential, which results in the application of electric current that delivers or enhances delivery of the agent, or, for “reverse” electrotransport, samples or enhances sampling of the agent. The electrotransport of the agents into or out of the human body can by achieved in various manners.

One widely used electrotransport process, iontophoresis, involves the electrically induced transport of charged ions. Electroosmosis, another type of electrotransport process involved in the transdermal transport of uncharged or neutrally charged molecules (e.g., transdermal sampling of glucose), involves the movement of a solvent with the agent through a membrane under the influence of an electric field. Electroporation, still another type of electrotransport, involves the passage of an agent through pores formed by applying an electrical pulse, a high voltage pulse, to a membrane.

In many instances, more than one of the noted processes may be occurring simultaneously to different extents. Accordingly, the term “electrotransport” is given herein its broadest possible interpretation, to include the electrically induced or enhanced transport of at least one charged or uncharged agent, or mixtures thereof, regardless of the specific mechanism(s) by which the agent is actually being transported. Additionally, other transport enhancing methods such as sonophoresis or piezoelectric devices can be used in conjunction with the invention.

### EXAMPLES

The following examples are given to enable those skilled in the art to more clearly understand and practice the present invention. They should not be considered as limiting the scope of the invention but merely as being illustrated as representative thereof.

### Example 1

Experiments were performed on a hairless guinea pig to evaluate agent delivery from high payload microprojection systems of the dual thrombin/factor Xa inhibitor RWJ-445167, which is under evaluation as a daily treatment anticoagulant for acute and venous thrombosis.

Pretreatment and integrated systems described In U.S. application Ser. Nos. 10/971,430 and 10/970,901 were employed for the following analysis. Microprojection designs 30a-30g, shown in FIG. 1, were analyzed. The microprojection designs with retention features (i.e., 30a-30c) were used with the integrated system while the microprojection designs without retention features (i.e., 30d-30g) were used with the pretreatment system.

The gel formulation employed with the integrated systems contained 20 wt % RWJ-445167 in an aqueous gel containing 50 wt % propylene glycol and 3% HEC. Following application of the systems, the gel formulation was left in contact with the skin for up to 24 h. Urine was collected for 24 h after removal of the formulation and intact RWJ-445167 was measured by LC-MS. Total amounts of drug excreted in urine were calculated and total amounts transported were extrapolated using urinary excretion results obtained following IV injection of RWJ-445167 (7.5% of the dose was found excreted intact in urine following injection of 0.5 to 3 mg RWJ-445167).

Referring now to FIG. 4, there is shown a series of photographs of the skin site following 24 h system wearing time. The photographs demonstrate that irritation and bleeding is minimized with shorter microprojection length.

An additional experiment using the pretreatment system was performed to compare microprojections lengths of 120 and 145 μm. The skin site photographs shown in FIG. 5 demonstrate that some pinpoint bleeding is still observed with the 145 μm microprojection length, while no bleeding or erythema was observed with the 120 μm microprojection length.

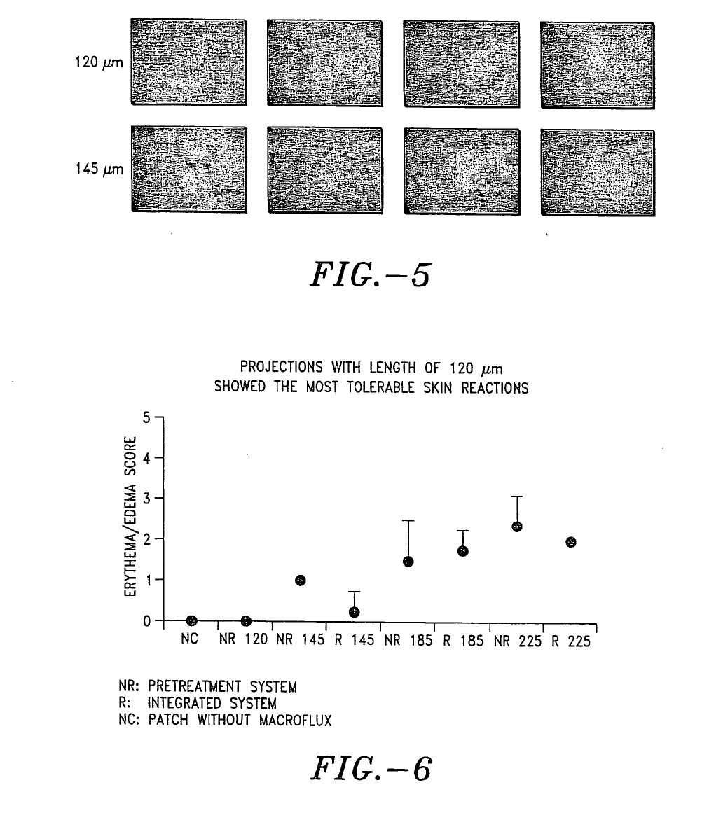

Referring now to FIG. 6, there is shown a graph illustrating the combined erythema+edema score obtained with the various microprojection designs. As illustrated in FIG. 6, absence of irritation was only observed with the 120 μm microprojections. The results thus confirm that lower irritation is observed with shorter microprojections. Further, the skin site was not distinguishable from control sites (24 h wearing with the same formulation, no microprojection treatment).

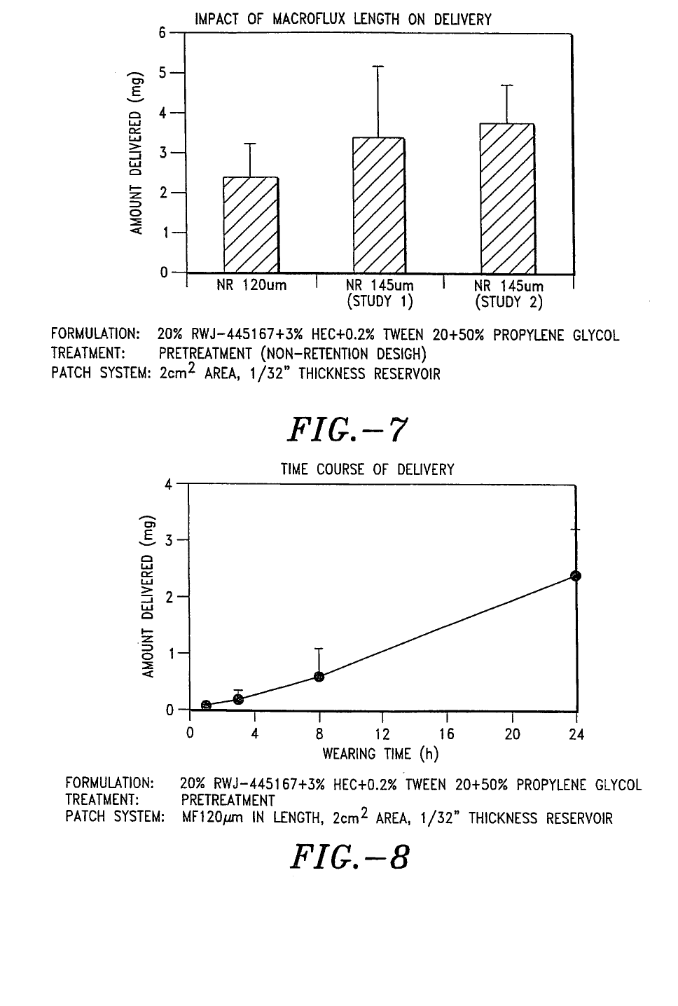

Evaluation of drug absorption following 24 h wearing demonstrated only a slight decrease in drug absorption with decreased microprojection length (see FIG. 7). Drug absorption was not measurable (detection limit=0.2 μg absorbed per 24 h) when the formulation was applied to the skin for 24 h in the absence of treatment with a microprojection array. This demonstrates that the microprojection treatment is very effective as about 2.5 mg of drug were absorbed following pretreatment with a 120 μm microprojection array, which corresponds to an enhancement factor of at least 10000 fold. In addition, sustained delivery for 24 h was achieved with the 120 μm microprojections (see FIG. 8).

The noted example thus demonstrates that optimal biocompatibility is achieved using a 120 μm microprojection array with acceptable agent delivery.

From the foregoing description, one of ordinary skill in the art can easily ascertain that the present invention, among other things, provides an effective and efficient means for enhancing the biocompatibility transdermal delivery systems.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A device for transdermally delivering a pharmacologically active agent, the device comprising:
a member having a plurality of stratum corneum-piercing microprojections, each of the microprojections having a length less than approximately 145 microns; and
at least one active agent adapted to be delivered transdermally by the microprojections.

2. The device of claim 1, wherein the microprojections have a length in the range of approximately 50 to 145 microns.

3. The device of claim 1, wherein the microprojections have a length in the range of approximately 70 to 140 microns.

4. The device of claim 1, wherein the microprojections have a length of about 120 microns.

5. The device of claim 1, wherein the microprojections are arranged in an array having a microprojection density greater than 100 microprojections/cm².

6. The device of claim 1, wherein the microprojections are arranged in an array having a microprojection density in the range of approximately 200 to 3000 microprojections/cm² .

7. The device of claim 1, wherein the microprojections are manufactured from a metal consisting of the group of stainless steel, titanium, nickel titanium alloys, or similar biocompatible materials.

8. The device of claim 1, wherein the active agent is coated on at least one of the microprojections.

9. The device of claim 1, wherein the active agent is in a biocompatible coating disposed on the at least one microprojection.

10. The device of claim 1, wherein the active agent is selected from the group consisting of small molecular weight compounds, polypeptides, proteins, oligonucleotides, nucleic acids and polysaccharides.

11. The device of claim 1, wherein the microprojections do not produce appreciable bleeding or irritation when applied to the skin of a subject.

12. A method of transdermally delivering a pharmacologically active agent, the method comprising:
providing a delivery system having a microprojection member that includes a plurality of stratum corneum-piercing microprojections, wherein each of the microprojections has a length less than approximately 145 microns; and at least one active agent adapted to be delivered transdermally by the microprotrusions; and
applying the member to a surface of a subject.

13. The method of claim 12, wherein the microprojections have a length in the range of approximately 50 to 145 microns.

14. The method of claim 12, wherein the microprojections have a length in the range of approximately 70 to 140 microns.

15. The method of claim 12, wherein the microprojections have a length of about 120 microns.

16. The method of claim 12, wherein the microprojections are arranged in an array having a microprojection density greater than 100 microprojections/cm².

17. The method of claim 12, wherein the microprojections are arranged in an array having a microprojection density in the range of approximately 200 to 3000 microprojections/cm².

18. The method of claim 12, wherein the microprojections do not produce appreciable bleeding or irritation when applied to the skin of a subject.
